(19) United States
(12) Patent Application Publication
(10) Pub. No.: US 2023/0201525 A1
(43) Pub. Date: Jun. 29, 2023

(54) CATHETER

(71) Applicant: ASAHI INTECC CO., LTD., Seto-shi (JP)

(72) Inventor: Kenji KANEDA, Seto-shi (JP)

(73) Assignee: ASAHI INTECC CO., LTD., Seto-shi (JP)

(21) Appl. No.: 18/118,405

(22) Filed: Mar. 7, 2023

Related U.S. Application Data
(63) Continuation of application No. PCT/JP2021/024965, filed on Jul. 1, 2021.

(30) Foreign Application Priority Data
Oct. 1, 2020 (JP) 2020-167018

(51) Int. Cl. A61M 25/00

(52) U.S. Cl. CPC A61M 25/005; A61M 25/0045

(57) ABSTRACT

A catheter includes a hollow shaft, a linear member provided on an outer periphery of the hollow shaft, and a metallic film that is formed between the outer periphery of the hollow shaft and the linear member and is joined to the outer periphery of the hollow shaft and the linear member. With this configuration, as the hollow shaft, the metallic film, and the linear member are joined together, the torqueability of the catheter may be improved.

### CROSS-REFERENCE TO RELATED APPLICATIONS

This is a Continuation of Application No. PCT/JP2021/024965 filed Jul. 1, 2021, which claims priority to JP 2020-167018 filed Oct. 1, 2020. The disclosure of the prior applications is hereby incorporated by reference herein in its entirety.

### TECHNICAL FIELD

The disclosed embodiments relate to a catheter.

### BACKGROUND ART

There is a conventionally disclosed catheter including an inner layer made of resin and a coil layer including a metallic wire on the outer side of the inner layer (see, for example, Patent Literature 1). Furthermore, there is a disclosed catheter including an inner tube made of resin and a braided body including a plurality of metallic wires on the outer side of the inner tube (see, for example, Patent Literature 2).

### CITATION LIST

### Patent Literature

Patent Literature 1: Japanese Patent No. 5075632

Patent Literature 2: JP H5-84303 A

### SUMMARY

### TECHNICAL PROBLEM

However, even with the above techniques, there is still room to improve torqueability in catheters in which a reinforcing body is formed on the outer side of the inner layer.

The disclosed embodiments have been made to solve at least a part of the above issues and have an object to improve the torqueability of catheters.

### SOLUTION TO PROBLEM

The disclosed embodiments have been made to solve the above issues and may be implemented as the aspects below.

(1) According to an aspect of the disclosed embodiments, a catheter is provided. A catheter includes a hollow shaft, a linear member provided in a linear manner on an outer periphery of the hollow shaft, and a metallic film that is formed between the outer periphery of the hollow shaft and the linear member and is joined to the outer periphery of the hollow shaft and the linear member.

With this configuration, as the hollow shaft, the metallic film, and the linear member are joined together, the torqueability of the catheter may be improved.

(2) In the catheter according to the above aspect, the linear member may be wound around the outer periphery of the hollow shaft in a spiral manner. With this configuration, as the linear member is shaped like a coil, the flexibility and torqueability of the catheter may be improved.

(3) In the catheter according to the above aspect, a height of the linear member may be larger than a width in transverse cross-section. With this configuration, the pressure resistance of the catheter may be improved as compared with the case where the height of the linear member is smaller than the width in transverse cross-section of the linear member.

(4) In the catheter according to the above aspect, the linear member may have a width that becomes smaller from an inner side where the linear member is joined to the metallic film toward an outer side in transverse cross-section. With this configuration, the volume of the linear member near the surface of the hollow shaft may be reduced while the height of the linear member is maintained. Accordingly, the flexibility near the surface of the hollow shaft is improved while the pressure resistance of the hollow shaft is maintained, and thus the catheter may be less likely to damage blood vessels or internal organs when the catheter is inserted into the body of the patient.

(5) In the catheter according to the above aspect, the metallic film may include stainless steel, and the linear member may include a nickel-cobalt alloy. With this configuration, the linear member made of a nickel-cobalt alloy may be formed on the outer peripheral surface of the metallic film by electrolytic plating using the metallic film made of stainless steel as a conductive film. As the linear member includes a nickel-cobalt alloy, the torqueability of the catheter may be improved.

(6) In the catheter according to the above aspect, the hollow shaft may include an inner layer made of PTFE (polytetrafluoroethylene) and an intermediate layer that is provided on an outer periphery of the inner layer and is made of PAE (thermoplastic polyamide elastomer). With this configuration, for example, the inner layer defines the lumen of the catheter and the inner layer is made of PTFE, and therefore it is possible to reduce the frictional resistance between the inner peripheral surface of the catheter and the device inserted into the inside of the catheter. Furthermore, as the intermediate layer includes PAE, the flexibility of the catheter and the restorability from curving of the catheter may be improved.

(7) In the catheter according to the above aspect, the linear member may be formed in a grid pattern on the outer periphery of the hollow shaft. With this configuration, the catheter including the metallic reinforcing body may be fabricated without using a metallic wire. Accordingly, there is no degradation in the flexibility of the catheter due to the interference between metallic wires at the overlapped portion of the metallic wires, and thus the flexibility of the catheter may be improved.

Moreover, the disclosed embodiments may be implemented in various modes, such as guide wires, guide wire manufacturing methods, endoscopes, dilators, etc.

### BRIEF DESCRIPTION OF DRAWINGS

FIG. 1 is an explanatory diagram illustrating an overall configuration of a catheter according to a first embodiment.

FIG. 2 is an explanatory diagram illustrating a partial cross section of the catheter according to the first embodiment.

FIG. 3 is an explanatory diagram of an enlarged region X1 in FIG. 2.

FIG. 4 is an explanatory diagram of an enlarged region Y1 in FIG. 2.

FIG. 5 is an explanatory diagram illustrating a cross-sectional configuration of the catheter according to the first embodiment.

FIG. 6 is an explanatory diagram of an enlarged region Z in FIG. 5.

FIG. 7 is an explanatory diagram illustrating a first manufacturing method of the catheter according to the first embodiment.

FIG. 8 is an explanatory diagram illustrating a partial cross section of the catheter according to a second embodiment.

FIG. 9 is an explanatory diagram of an enlarged region X2 in FIG. 8.

FIG. 10 is an explanatory diagram of an enlarged region Y2 in FIG. 8.

### DETAILED DESCRIPTION OF EMBODIMENTS

### First Embodiment

Catheters according to the disclosed embodiments will be described with reference to the drawings. The present disclosure is not limited to the embodiments described in the drawings.

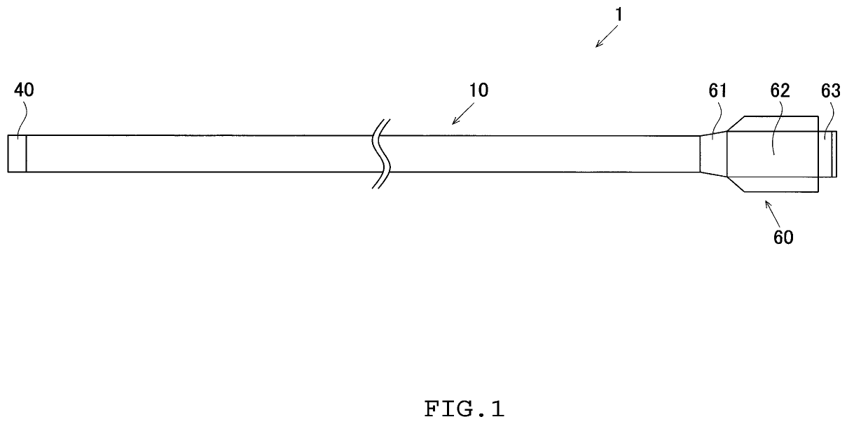

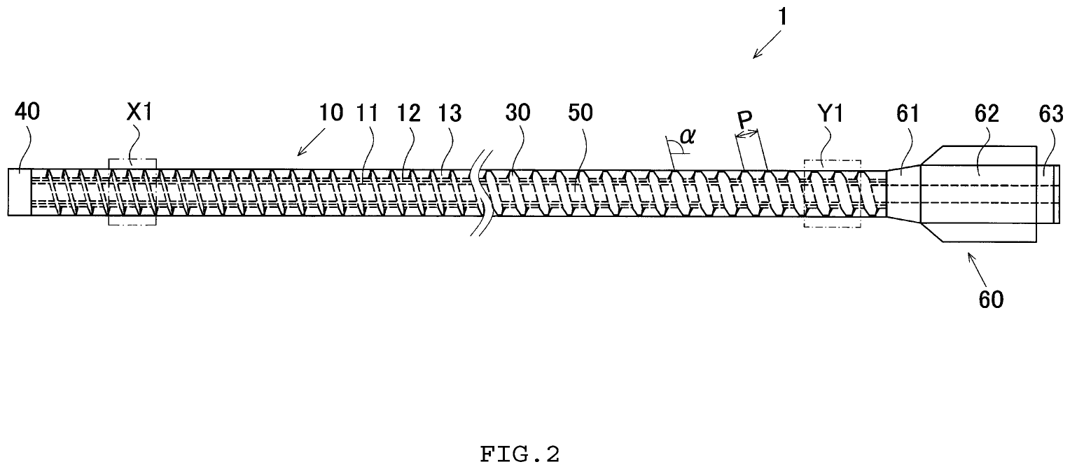

FIG. 1 is an explanatory diagram illustrating an overall configuration of a catheter 1 according to a first embodiment. FIG. 2 is an explanatory diagram illustrating a partial cross section of the catheter 1 (in which the catheter 1 can be considered “partially transparent”). FIG. 2 is a diagram illustrating a form of a linear member 30 in the entire catheter 1 that is partially transparent. In FIG. 2, a region X1 represents part of a distal end portion of a hollow shaft 10, and a region Y1 represents part of a proximal end portion of the hollow shaft 10.

In FIG. 1, the left side is the distal end side of the catheter 1 and each component of the catheter 1, and the right side is the proximal end side of the catheter 1 and each component of the catheter 1. The distal end side of the catheter 1 is the side (distal side) inserted into the body, and the proximal end side of the catheter 1 is the side (near side) operated by a technician such as a physician. The right-and-left direction in FIG. 1 is referred to as the axial direction of the catheter 1 and each component. The direction orthogonal to the axial direction is referred to as the radial direction of the catheter 1 and each component.

Furthermore, the end portion located on the distal end side of the catheter 1 and each component of the catheter 1 is described as “distal end”, and the portion that includes the “distal end” and extends to the middle from the distal end toward the proximal end side is described as “distal end portion”. Similarly, the end portion located in the proximal end side of the catheter 1 and each component of the catheter 1 is described as “proximal end”, and the portion that includes the “proximal end” and extends to the middle from the proximal end toward the distal end side is described as “proximal end portion”.

For convenience of explanation, FIG. 1 includes portions where the components are illustrated at the relative ratio of the size different from the actual relative ratio. The same applies to the explanatory diagrams illustrated in FIGS. 2 to 10.

The catheter 1 is a medical tool that is inserted into blood vessels and digestive organs for treatment and examination. The catheter 1 includes the hollow shaft 10, a distal tip 40, and a grip portion 60.

The hollow shaft 10 is an elongated tubular member extending in the axial direction of the catheter 1. The distal tip 40 is a tubular member joined to the distal end portion of the hollow shaft 10. The grip portion 60 is a tubular member that is joined to the proximal end portion of the hollow shaft 10 and is gripped by a technician such as a physician to operate the catheter 1.

The distal tip 40 is a tubular member joined to the distal end portion of the hollow shaft 10 to form part of a distal end portion of a lumen 50 of the catheter 1. The distal tip 40 may be made of a resin material having flexibility. For example, TPU (thermoplastic polyurethaneelastomer) is selectable. The distal tip 40 is not limited to resin materials, but may be made of a metallic material.

The grip portion 60 is a tubular member joined to the proximal end portion of the hollow shaft 10 to form part of a proximal end portion of the lumen 50 of the catheter 1. The grip portion 60 includes a protector 61, a main body portion 62, and a connector 63. The protector 61 has a tapered shape with the outer diameter increasing toward the proximal end side of the protector 61. The main body portion 62 includes a projection on its outer periphery to facilitate gripping by a technician such as a physician. The connector 63 is threaded on its inner peripheral side so as to be coupled to other medical devices such as a syringe (not illustrated). The grip portion 60 may be made of a material that is durable and suitable for sterilization processing. For example, a metal, an injection-molded resin, or a combination thereof may be used.

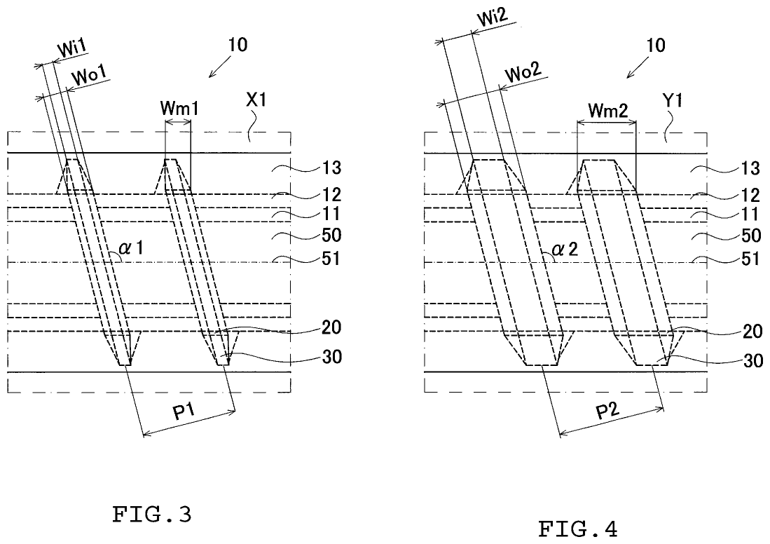

FIG. 3 is an explanatory diagram of the enlarged region X1 in FIG. 2. FIG. 3 is a diagram illustrating a form of a metallic film 20 and the linear member 30 in the partially enlarged, partially transparent distal end portion of the hollow shaft 10. FIG. 4 is an explanatory diagram of the enlarged region Y1 in FIG. 2. FIG. 4 is a diagram illustrating a form of the metallic film 20 and the linear member 30 in the partially enlarged, partially transparent proximal end portion of the hollow shaft 10.

The hollow shaft 10 includes an inner layer 11, an intermediate layer 12, an outer layer 13, the metallic film 20, and the linear member 30.

The inner layer 11 is an elongated tubular member provided inside the hollow shaft 10. The inner layer 11 defines the lumen 50 of the catheter 1. The intermediate layer 12 is an elongated tubular member covering the outer periphery of the inner layer 11. The outer layer 13 is an elongated tubular member provided outside the hollow shaft 10 to cover the outer periphery of the intermediate layer 12. The respective distal end portions of the inner layer 11, the intermediate layer 12, and the outer layer 13 are joined to the distal tip 40. The respective proximal end portions of the inner layer 11, the intermediate layer 12, and the outer layer 13 are joined to the grip portion 60.

To insert a medical device such as a guide wire (not illustrated) into the inside of the inner layer 11, the inner layer 11 may be made of a resin material having desirable slidability. For example, fluorine resins such as PTFE (polytetrafluoroethylene), PFA (tetrafluoroethylene-perfluoroalkyl vinyl ether copolymer) or FEP (tetrafluoroethylene-hexafluoropropylene copolymer), polyethylene, etc. is selectable. The inner layer 11 may be made of known materials other than those described above.

The intermediate layer 12 and the outer layer 13 may be made of an elastomer resin material, although not limited thereto in particular. For example, it may be made of PAE (thermoplastic polyamide elastomer), TPU (thermoplastic polyurethane elastomer), or TPEE (polyester elastomer), etc. The intermediate layer 12 and the outer layer 13 may be made of known materials other than those described above.

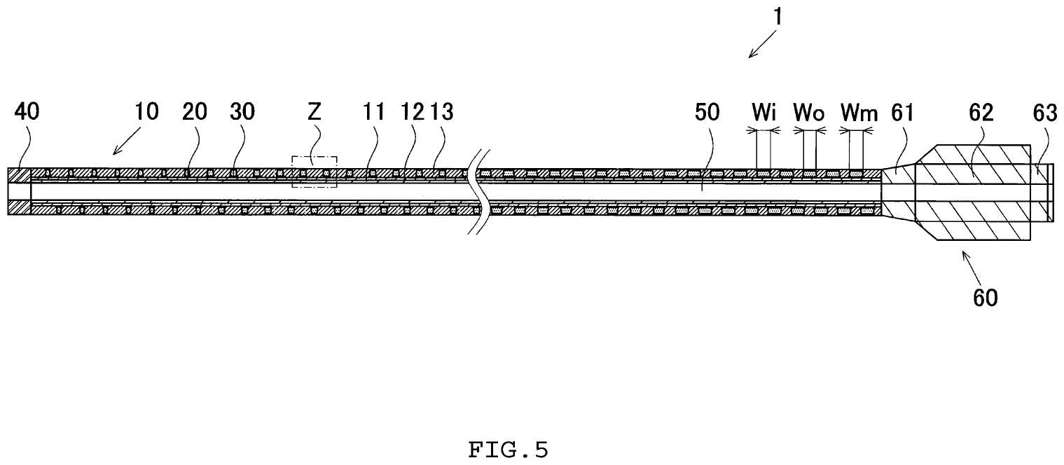

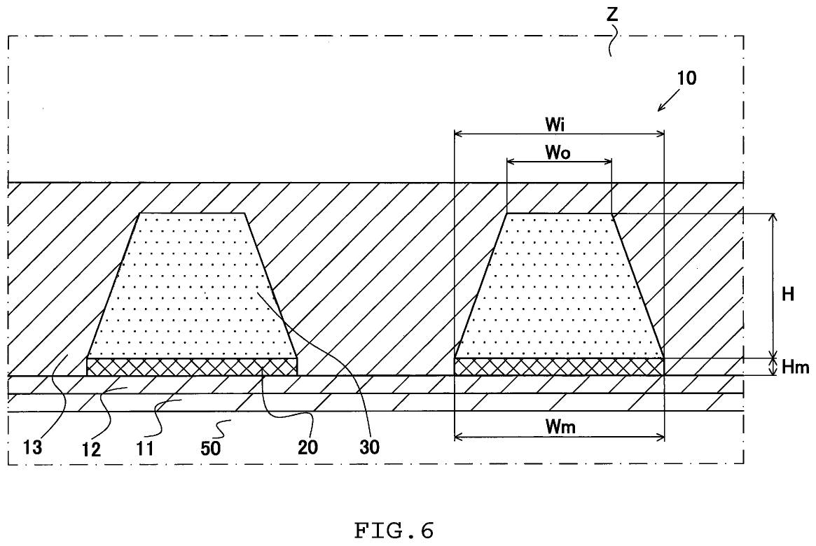

FIG. 5 is an explanatory diagram illustrating a cross-sectional configuration of the catheter according to the first embodiment. FIG. 5 is a cross-sectional view of the catheter 1 in longitudinal cross-section. In FIG. 5, a region Z represents a portion of the hollow shaft 10. FIG. 6 is an explanatory diagram of the enlarged region Z in FIG. 5. FIG. 6 is a diagram illustrating a form of the metallic film 20 and the linear member 30 in the partially enlarged hollow shaft in longitudinal cross-section of the catheter 1.

The metallic film 20 will be described using FIGS. 2 to 6. The metallic film 20 is a metallic film-like member formed on a surface of the intermediate layer 12 in a spiral manner along the axial direction of the catheter 1. The metallic film 20 is joined to the intermediate layer 12 and does not move relative to the intermediate layer 12 in the axial direction of the catheter 1. The size of the metallic film 20 in the axial direction of the catheter 1 is a width Wm of the metallic film 20. The width Wm of the metallic film 20 located in the region X1 is Wm1 as illustrated in FIG. 3, and the width of the metallic film 20 located in the region Y1 is Wm2 as illustrated in FIG. 4. As illustrated in FIG. 5, the width Wm of the metallic film 20 becomes smaller toward the distal end of the catheter 1. In other words, the width Wm1 of the metallic film 20 located at the distal end side is smaller than the width Wm2 of the metallic film 20 located in the proximal end portion of the catheter 1. The height of the metallic film 20 is Hm. The height Hm of the metallic film 20 may be set, for example, from 1 Å (0.1 nm) to 100 Å (10 nm). The metallic film 20 may be made of stainless steel, for example. Alternatively, it may also be made of silver, copper, etc. As silver and copper have high electric conductivity, it is relatively easy to study the conditions for various patterns. In particular, silver is effective in coating for areas that need blood contact because of its high electric conductivity and high performance in terms of biological compatibility.

The linear member 30 will be described using FIGS. 2 to 6. As illustrated in FIG. 2, the linear member 30 is a metallic linear member formed on a surface of the metallic film 20 in a spiral manner along the axial direction of the catheter 1. The linear member 30 is joined to the metallic film 20 and does not move relative to the metallic film 20 in the axial direction of the catheter 1. When the catheter 1 is observed from a plane (longitudinal cross-section) parallel to the axial direction of the catheter 1, the linear member 30 repeatedly appears across the axis of the catheter 1. In the repeatedly appearing linear member 30, the interval between the adjacent linear members 30 is a pitch P of the linear member 30, and the angle of each of the linear members 30 with respect to a virtual axis 51 of the catheter 1 is an inclination angle α. The pitch P of the linear member 30 becomes smaller toward the distal end of the catheter 1. The inclination angle a of the linear member 30 becomes smaller toward the distal end of the catheter 1.

The pitch P of the linear member 30 located in the region X1 is P1 as illustrated in FIG. 3, and the pitch P of the linear member 30 located in the region Y1 is P2 as illustrated in FIG. 4. The pitch P1 of the linear member 30 located at the distal end side is smaller than the pitch P2 of the linear member 30 located in the proximal end portion of the catheter 1. The linear member 30 is joined to the metallic film 20, and the metallic film 20 is joined to the intermediate layer 12, and therefore the pitch P does not change unless the intermediate layer 12 is deformed due to curving, etc. The inclination angle α of the linear member 30 located in the region X1 is α1, and the inclination angle α of the linear member 30 located in the region Y1 is α2. The inclination angle α1 of the linear member 30 located at the distal end side is smaller than the inclination angle α2 of the linear member 30 located in the proximal end portion of the catheter 1.

As illustrated in FIGS. 5 and 6, the linear member 30 is trapezoidal in longitudinal cross-section of the catheter 1. The linear member 30 has a width Wi of a portion in contact with the metallic film 20 and a width Wo of a portion near the outer peripheral surface of the catheter 1. The width Wi is larger than the width Wo. The width Wi and the width Wo become smaller toward the distal end of the catheter 1. The width Wi of the linear member 30 located in the region X1 is Wi1 and the width Wo is Wo1 as illustrated in FIG. 3, and the width Wi of the linear member 30 located in the region Y1 is Wi2 and the width Wo is Wo2 as illustrated in FIG. 4. The linear member 30 has a height H. The height H is larger than the width Wi and, for example, the ratio of the height H to the width Wi may be set to 3 to 1. The height H of the linear member 30 may be set, for example, from 10 μm to 100 μm. The height H is substantially constant in the axial direction of the catheter 1. The linear member 30 may be made of a nickel-cobalt alloy, for example.

### Effect Example

With this configuration, joining the hollow shaft 10, the metallic film 20, and the linear member 30 may improve torqueability, pressure resistance, elongation resistance, etc., of the catheter 1. It is possible to reduce the rate of attenuation of the torque, which is generated in the catheter 1 due to the rotation of the grip portion 60 by a technician such as a physician, from the proximal end portion to the distal end portion of the catheter 1. Furthermore, when the linear member 30 is simply wound around the hollow shaft 10 in a spiral manner and the linear members 30 are not joined to the hollow shaft 10, the end portion of the linear member 30 is likely to expand in the radial direction due to its own elasticity. With this configuration, as the metallic film 20 and the linear member 30 are joined to the hollow shaft 10, the end portion of the linear member 30 may be less likely to expand in the radial direction.

Further, the hollow shaft of the catheter may include a side hole communicating with the inside and the outside of the hollow shaft to inject a medicinal solution such as contrast medium. In this case, when a coil body formed by winding a metallic wire around the hollow shaft in a spiral manner or a braided body formed by braiding a plurality of metallic wires is used as a reinforcing body, the hollow shaft receives a restoring force with which the metallic wire of the coil body or the braided body returns to a linear shape. This may cause a damage to the hollow shaft around the side hole serving as a base point. With this configuration, no metallic wire is provided, and the linear member 30 is joined to the intermediate layer 12 via the metallic film 20, and therefore no pressure is applied from the linear member 30 to the hollow shaft 10. Thus, even when the catheter 1 includes the side hole, the hollow shaft 10 may be less likely to get damaged at the side hole serving as a base point.

Furthermore, the width Wm of the metallic film 20 and the width Wi and the width Wo of the linear member 30 become smaller toward the distal end of the catheter 1, and thus the catheter 1 has a structure in which the flexibility increases toward the distal end. When the catheter 1 is inserted into the body of the patient and the catheter 1 comes into contact with a blood vessel or internal organ of the patient, the blood vessel or internal organ of the patient is less likely to get damaged. Moreover, when the catheter 1 is inserted into a curved blood vessel, or the like, the catheter 1 is deformed along the curved shape of the blood vessel so that the catheter 1 may proceed through the blood vessel.

In transverse cross-section of the linear member 30, the height H is larger than the width Wi, and thus the durability against the pressure applied in the radial direction of the hollow shaft 10 is improved as compared with the case where the height H is smaller than the width Wi. Further, the width Wi is smaller than the height H, and thus the gap between the linear members 30 in the axial direction of the catheter 1 is relatively large. This results in a relatively large volume of the resin layers, such as the inner layer 11, the intermediate layer 12, and the outer layer 13, provided in the gap. Therefore, the resin layer may reduce the compressive force or the tensile force in the axial direction of the catheter 1 applied to the outer peripheral surface of the catheter 1 when the catheter 1 is curved. Moreover, the large height H of the linear member 30 increases the resistance against the force applied in the radial direction of the catheter 1, and thus deformation is suppressed. The above allows the catheter 1 to maintain its roundness during operation. Furthermore, in transverse cross-section of the linear member 30, the width Wi is larger than the width Wo, and thus the volume of the linear member on the surface portion of the hollow shaft 10 may be reduced. This may improve the flexibility near the surface of the hollow shaft 10 while maintaining the pressure resistance of the hollow shaft 10. Thus, when the catheter 1 comes into contact with a blood vessel, internal organ, or the like, of the patient, the catheter 1 is less likely to damage the blood vessel or internal organ of the patient.

When the metallic film 20 includes stainless steel and the linear member 30 includes a nickel-cobalt alloy, the linear member 30 may be formed on the outer peripheral surface of the metallic film 20 by electrolytic plating using the metallic film 20 as a conductive film. As the linear member 30 includes a nickel-cobalt alloy, the catheter 1 having desirable torqueability may be fabricated. Furthermore, as the inner layer 11 includes PTFE, it is possible to reduce the frictional resistance between the inner peripheral surface of the catheter 1 and the device inserted into the inside of the catheter 1. As the intermediate layer 12 or the outer layer 13 includes PAE, the flexibility of the catheter 1 and the restorability from curving may be improved.

As the inclination angle α of the linear member 30 becomes smaller toward the distal end of the catheter 1 and the pitch P becomes smaller toward the distal end of the catheter 1, the linear member 30 may be densely formed toward the distal end of the catheter 1. This improves torqueability to the distal end of the catheter 1.

### Manufacturing Method

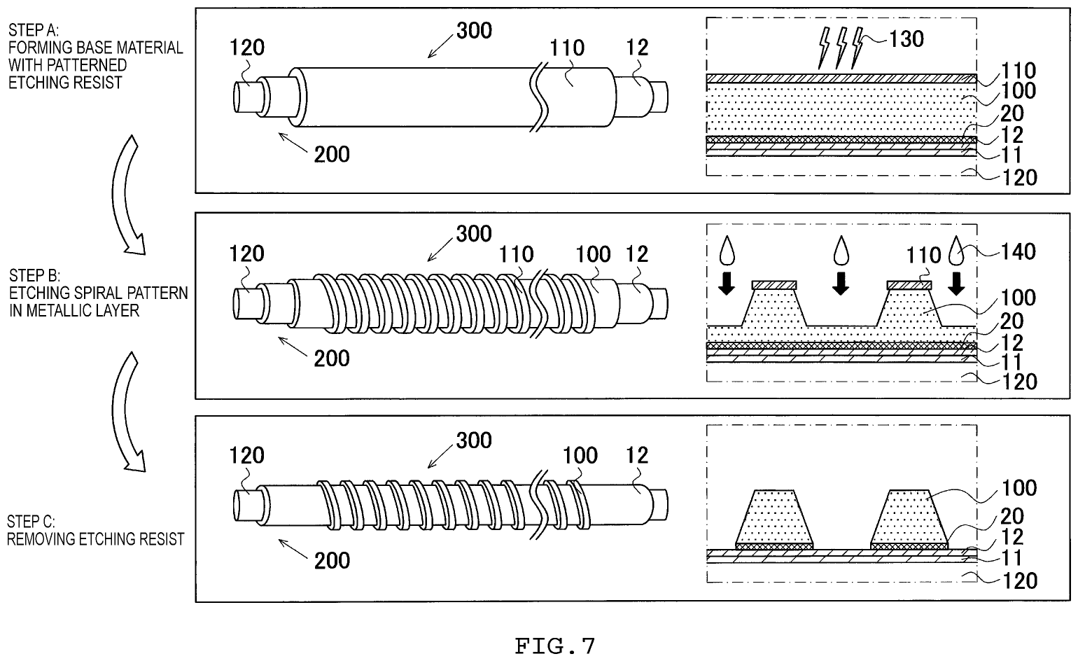

FIG. 7 is an explanatory diagram illustrating a first manufacturing method of the catheter 1 according to the first embodiment.

First, as illustrated in Step A, a core material 120 is coated with the inner layer 11 and the intermediate layer 12 by extrusion molding, or the like, to produce a hollow shaft 200. Subsequently, the outer periphery of the intermediate layer 12 is coated with the metallic film 20 by electroless plating, sputtering, or the like, and the outer periphery of the metallic film 20 is coated with a metallic layer 100 by electrolytic plating, or the like. Subsequently, etching resist 110 made of etching-resistant resin is applied to the outer periphery of the metallic layer 100. By the above process, a base material 300 is fabricated. Subsequently, for example, while the base material 300 is rotated around the axial direction of the base material 300 and moved in the axial direction, the outer periphery of the base material 300 is irradiated with a laser 130. This removes the etching resist 110 in a spiral manner. Subsequently, as illustrated in Step B, the metallic layer 100 exposed to the outside in a spiral manner due to the removal of the etching resist 110 and the metallic film 20 on the inner side are melted by etchant 140. At this time, the metallic layer 100 begins to melt from a portion of the metallic layer 100 located on the outer side in the radial direction. Therefore, the portion of the metallic layer 100 located on the outer side in the radial direction has a larger amount of melting, and the portion located on the inner side in the radial direction has a smaller amount of melting. Accordingly, in longitudinal cross-section of the catheter 1, the molten metallic layer 100 is trapezoidal with the width increasing from the outer side in the radial direction to the inner side in the radial direction. Subsequently, as illustrated in Step C, the etching resist 110 is removed, which has not been removed by the laser 130 during the previous process and remains on the outer periphery of the metallic layer 100 formed in a spiral manner. The above process may fabricate the base material 300 in which the outer periphery of the core material 120 is coated with the inner layer 11 and the intermediate layer 12, and the metallic film 20 and the metallic layer 100 are formed in a spiral manner on the outer periphery of the intermediate layer 12. The base material 300 may be coated with the outer layer 13 (see FIGS. 1 to 6).

### Effect Example

With this configuration, the catheter 1 including the reinforcing body made of metal may be manufactured without using a metallic wire. The metallic film 20 is formed on the outer surface of the hollow shaft by electroless plating, sputtering, or the like, and the metallic layer 100 is formed on the outer surface of the metallic film 20 by electrolytic plating, or the like. Accordingly, the metallic layer 100 is joined to the intermediate layer 12 through the metallic film 20. Thus, for example, when the outer periphery of the metallic layer 100 is coated with a resin tube to form the outer layer 13, the position of the metallic layer 100 is not moved relative to the hollow shaft 200. This improves the manufacturing efficiency of the catheter 1.

### Second Embodiment

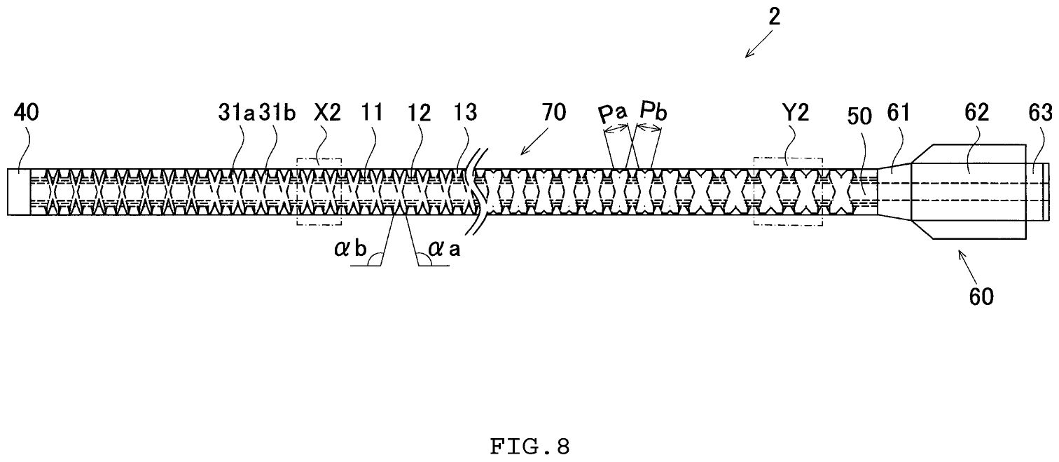

FIG. 8 is an explanatory diagram illustrating an overall configuration of a partially transparent catheter 2 according to a second embodiment. FIG. 8 is a diagram illustrating a form of a linear member 31 in the entire catheter 2 that is partially transparent. The catheter 2 according to the second embodiment includes a hollow shaft 70 instead of the hollow shaft 10 of the catheter 1 according to the first embodiment. The hollow shaft 70 includes a metallic film 21 instead of the metallic film 20 in the catheter 1 and the linear member 31 instead of the linear member 30. The catheter 2 is identical to the catheter 1 except for the portions other than the hollow shaft 70. Therefore, the descriptions of the members other than the hollow shaft 70 are omitted. A region X2 represents part of a distal end portion of the hollow shaft 70, and a region Y2 represents part of a proximal end portion of the hollow shaft 70.

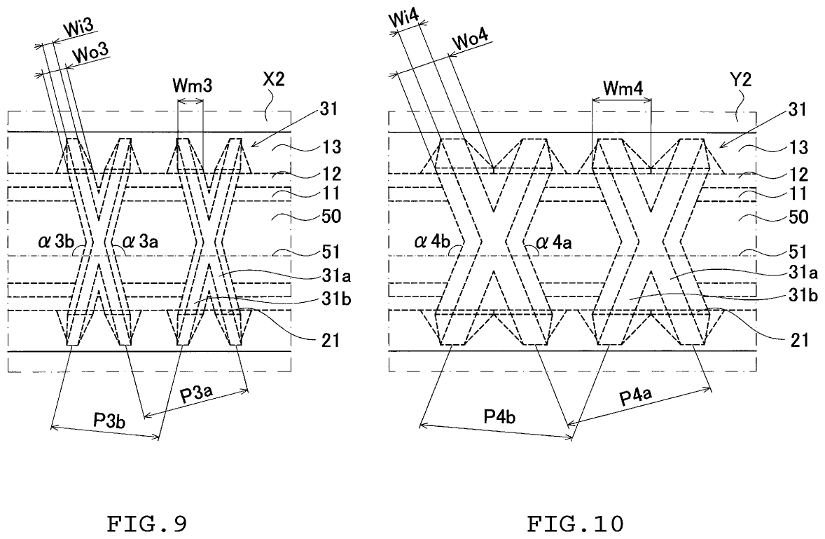

FIG. 9 is an explanatory diagram of the enlarged region X2 in FIG. 8. FIG. 9 is a diagram illustrating a form of the metallic film 21 and the linear member 31 in the partially enlarged, partially transparent distal end portion of the hollow shaft 70. FIG. 10 is an explanatory diagram of the enlarged region Y2 in FIG. 8. FIG. 10 is a diagram illustrating a form of the metallic film 21 and the linear member 31 in the partially enlarged, partially transparent proximal end portion of the hollow shaft 70.

As illustrated in FIG. 8, the metallic film 21 is a metallic film-like member that is formed on the outer periphery of the intermediate layer 12 in a grid pattern along the axial direction of the catheter 2. The metallic film 21 is joined to the intermediate layer 12 and does not move relative to the intermediate layer 12 in the axial direction of the catheter 2. The width Wm of the metallic film 21 located in the region X2 is Wm3 as illustrated in FIG. 9, and the width of the metallic film 21 located in the region Y2 is Wm4 as illustrated in FIG. 10. In this case, the relation between Wm3 and Wm4 is similar to the relation between Wm1 and Wm2 according to the first embodiment.

As illustrated in FIG. 8, the linear member 31 is a metallic linear member that is formed on the outer periphery of the metallic film 21 in a grid pattern along the axial direction of the catheter 2. The linear member 31 is joined to the metallic film 21 and does not move relative to the metallic film 21 in the axial direction of the catheter 2. When the catheter 2 is observed from a plane (longitudinal cross-section) parallel to the axial direction of the catheter 2, the X-shaped linear member 31 repeatedly appears across the axis of the catheter 2. In longitudinal cross-section of the catheter 2, in the linear member 31, a portion tilting to the distal end side with respect to the axis of the catheter 2 is an element 31a of the linear member 31, and a portion tilting to the proximal end side is an element 31b. The element 31a and the element 31b are not separate members, but the linear member 31 is integrally formed over its entire length. Therefore, when the catheter 2 is operated by a technician such as a physician, the element 31a and the element 31b do not move independently, and thus the X-shape formed by the element 31a and the element 31b is maintained during the operation of the catheter 2.

As illustrated in FIG. 9, an inclination angle αa of the element 31a of the linear member 31 located in the region X2 with respect to the virtual axis 51 of the catheter 2 is α3a, and an inclination angle αb of the element 31b of the linear member 31 located in the region X2 with respect to the virtual axis 51 of the catheter 2 is α3b. As illustrated in FIG. 10, the inclination angle αa of the element 31a of the linear member 31 located in the region Y2 with respect to the virtual axis 51 of the catheter 2 is α4a, and the inclination angle αb of the element 31b of the linear member 31 located in the region Y2 with respect to the virtual axis 51 of the catheter 2 is α4b. In each of the linear members 31, the measures of the inclination angle αa and the inclination angle αb are substantially identical. In FIG. 9, the measures of the inclination angle α3a and the inclination angle α3b are substantially identical. In FIG. 10, the measures of the inclination angle α4a and the inclination angle α4b are substantially identical.

As illustrated in FIG. 9, a pitch Pa of the adjacent elements 31a of the linear member 31 located in the region X2 is P3a, and a pitch Pb of the adjacent elements 31b of the linear member 31 located in the region X2 is P3b. As illustrated in FIG. 10, the pitch Pa of the adjacent elements 31a of the linear member 31 located in the region Y2 is P4a, and the pitch Pb of the adjacent elements 31b of the linear member 31 located in the region Y2 is P4b. In each of the linear members 31, the measures of the pitch Pa and the pitch Pb are substantially identical. In FIG. 9, the measures of the pitch P3a and the pitch P3b are substantially identical. In FIG. 10, the measures of the pitch P4a and the pitch P4b are substantially identical.

As illustrated in FIG. 9, the width Wi of the linear member 31 located in the region X2 is Wi3, and the width Wo is Wo3. As illustrated in FIG. 10, the width Wi of the linear member 31 located in the region Y2 is Wi4, and the width Wo is Wo4. In this case, the relation between Wi3 and Wi4 is similar to the relation between Wi1 and Wi2 according to the first embodiment. Furthermore, the relation between Wo3 and Wo4 is similar to the relation between Wo1 and Wo2 according to the first embodiment.

### Effect Example

When the catheter includes a braided body that is formed by braiding a plurality of metallic wires, the curved catheter causes the metallic wires to interfere with each other at the intersection of the metallic wires. This causes the braided body to deform into an elliptical shape, which may reduce torqueability, pushing-pulling operation performance, and flexibility. However, the linear member 31 is an integrally formed grid-like member and does not include a plurality of metallic wires. Therefore, there is no interference between metallic wires, and the linear member 31 may maintain the shape similar to an exact circle even when the catheter 2 is curved. This may improve torqueability, pushing/pulling performance, and flexibility. Furthermore, the outer diameter of the catheter may be reduced as compared with the case where a plurality of metallic wires is used. This makes it easy to insert the catheter 2 into a peripheral blood vessel having a small inner diameter. Further, when the linear members 31 are simply wound around the hollow shaft 70 so as to be braided, and the linear members 31 and the hollow shaft 70 are not joined, the end portion of the linear member 31 is likely to expand in the radial direction due to its own elasticity. With this configuration, as the metallic film 21 and the linear member 31 are joined to the hollow shaft 70, the end portion of the linear member 31 may be less likely to expand in the radial direction.

The inclination angle αa of the element 31a of the linear member 31 is substantially identical to the inclination angle αb of the element 31b, and the pitch Pa of the element 31a is substantially identical to the pitch Pb of the element 31b so that the torqueability may be improved regardless of the rotation direction of the catheter 2.

### Modifications of Present Embodiment

The disclosed embodiments are not limited to the above embodiments, but may be implemented in various modes without departing from the gist thereof and may be modified as described below for example.

[Modification 1]

In the catheter 1 according to the first embodiment, the metallic film 20 is joined to the surface of the intermediate layer 12. However, the catheter 1 may omit the intermediate layer 12 and may include only the inner layer 11 and the outer layer 13 so that the metallic film 20 may be joined to the surface of the inner layer 11. Furthermore, the width Wm of the metallic film 20 becomes smaller toward the distal end of the catheter 1. However, the width Wm of the metallic film 20 may become larger toward the distal end of the catheter 1. In this case, the rigidity of the distal end portion of the catheter 1 may be increased.

[Modification 2]

In the catheter 1 according to the first embodiment, the pitch P of the linear member 30 becomes smaller toward the distal end of the catheter 1. However, the pitch P of the linear member 30 may become larger toward the distal end of the catheter 1. This may further improve the flexibility of the distal end portion of the catheter 1. Moreover, the inclination angle α of the linear member 30 becomes smaller toward the distal end of the catheter 1. However, the inclination angle α of the linear member 30 may become larger toward the distal end of the catheter 1.

[Modification 3]

In the catheter 1 according to the first embodiment, the linear member 30 is trapezoidal in longitudinal cross-section of the catheter 1. However, the linear member 30 may be formed in various forms, such as square, rectangular, or semicircular that is a shape protruding toward the outer side in the radial direction, instead of a trapezoidal shape in longitudinal cross-section of the catheter 1. Moreover, although the width Wi is larger than the width Wo, the width Wi may be smaller than the width Wo.

[Modification 4]

In the catheter 1 according to the first embodiment, the width Wi and the width Wo of the linear member 30 become smaller toward the distal end of the catheter 1. However, the width Wi and the width Wo of the linear member 30 may become larger toward the distal end of the catheter 1. This may increase the rigidity of the distal end portion of the catheter 1. Furthermore, the height H of the linear member 30 is larger than the width Wi. However, the height H of the linear member 30 may be smaller than the width Wi. In this case, the outer diameter of the catheter 1 may be smaller as compared to the case where the height H is larger than the width Wi. Moreover, the height H of the linear member 30 is substantially constant in the axial direction of the catheter 1; however, the height of the linear member 30 H does not need to be substantially constant in the axial direction of the catheter 1. For example, the height H may decrease toward the distal end of the catheter 1. In this case, the flexibility of the distal end portion of the catheter 1 may be further improved.

The above-described modifications of the catheter 1 according to the first embodiment may be applied to the second embodiment within an applicable range.

[Modification 5]

In the catheter 2 according to the second embodiment, the measures of the inclination angle αa and the inclination angle αb of each of the linear members 31 are substantially identical. However, the measures of the inclination angle αa and the inclination angle αb do not need to be substantially identical. In this case, the linear member 31 has an X-shape that is not linearly symmetrical when the catheter 2 is observed from a plane (longitudinal cross-section) parallel to the axial direction of the catheter 2.

[Modification 6]

In the catheter 2 according to the second embodiment, the measures of the pitch Pa and the pitch Pb of each of the linear members 31 are substantially identical. However, the measures of the pitch Pa and the pitch Pb do not need to be substantially identical. In this case, the linear member 31 has an X-shape that is not linearly symmetrical when the catheter 2 is observed from a plane (longitudinal cross-section) parallel to the axial direction of the catheter 2.

The present aspect has been described above based on the embodiments and the modifications; however, the embodiments according to the above aspects are provided to facilitate understanding of the present aspect and not to limit the aspect. The present aspect may be modified and improved without departing from the spirit thereof and the scope of claims, and the present aspect includes equivalents thereof. Furthermore, the technical features may be omitted as appropriate unless they are described as essential in this description.

### DESCRIPTION OF REFERENCE NUMERALS

1 Catheter according to first embodiment

2 Catheter according to second embodiment

10 Hollow shaft according to first embodiment

11 Inner layer

12 Intermediate layer

13 Outer layer

20 Metallic film according to first embodiment

21 Metallic film according to second embodiment

30 Linear member according to first embodiment

31 Linear member according to second embodiment

31a Portion tilting to distal end side of linear member according to second embodiment

31b Portion tilting to proximal end side of linear member according to second embodiment

40 Distal tip

50 Lumen

51 Virtual axis of catheter

60 Grip portion

61 Protector

62 Main body portion

63 Connector

70 Hollow shaft according to second embodiment

100 Metallic layer

120 Core material

130 Laser

140 Etchant

200 Hollow shaft

300 Base material

Wm Width of metallic film

Hm Height of metallic film

Wi Width of portion in contact with metallic film 20 of linear member

Wo Width on outer side of linear member

H Height of linear member

P Pitch of linear members

Pa Pitch of portion tilting to distal end side of linear member according to second embodiment

Pb Pitch of portion tilting to proximal end side of linear member according to second embodiment

α Inclination angle of linear member

αa Inclination angle of portion tilting to distal end side of linear member according to second embodiment

αb Inclination angle of portion tilting to proximal end side of linear member according to second embodiment

What is claimed is:

1. A catheter comprising:
a hollow shaft;
a linear member provided on an outer periphery of the hollow shaft; and
a metallic film that is positioned between the outer periphery of the hollow shaft and the linear member and is joined to the outer periphery of the hollow shaft and the linear member.

2. The catheter according to claim 1, wherein the linear member is wound around the outer periphery of the hollow shaft in a spiral manner.

3. The catheter according to claim 2, wherein a height of the linear member is larger than a width of the linear member in a transverse cross-section of the linear member.

4. The catheter according to claim 3, wherein the width of the linear member is larger at an inner side of the linear member in a radial direction where the linear member is joined to the metallic film than at an outer side of the linear member in the radial direction.

5. The catheter according to claim 4, wherein:
the metallic film includes stainless steel, and
the linear member includes a nickel-cobalt alloy.

6. The catheter according to claim 5, wherein the hollow shaft comprises:
an inner layer made of polytetrafluoroethylene (PTFE); and
an intermediate layer that is provided on an outer periphery of the inner layer and is made of thermoplastic polyamide elastomer (PAE).

7. The catheter according to claim 1, wherein the linear member is formed in a grid pattern on the outer periphery of the hollow shaft.

8. The catheter according to claim 1, wherein a height of the linear member is larger than a width of the linear member in a transverse cross-section of the linear member.

9. The catheter according to claim 8, wherein the width of the linear member is larger at an inner side of the linear member in a radial direction where the linear member is joined to the metallic film than at an outer side of the linear member in the radial direction.

10. The catheter according to claim 8, wherein:
the metallic film includes stainless steel, and
the linear member includes a nickel-cobalt alloy.

11. The catheter according to claim 8, wherein the hollow shaft comprises:
an inner layer made of PTFE; and
an intermediate layer that is provided on an outer periphery of the inner layer and is made of PAE.

12. The catheter according to claim 1, wherein a width of the linear member is larger at an inner side of the linear member in a radial direction where the linear member is joined to the metallic film than at an outer side of the linear member in the radial direction.

13. The catheter according to claim 12, wherein
the metallic film includes stainless steel, and
the linear member includes a nickel-cobalt alloy.

14. The catheter according to claim 12, wherein the hollow shaft comprises:
an inner layer made of PTFE; and
an intermediate layer that is provided on an outer periphery of the inner layer and is made of PAE.

15. The catheter according to claim 1, wherein
the metallic film includes stainless steel, and
the linear member includes a nickel-cobalt alloy.

16. The catheter according to claim 15, wherein the hollow shaft comprises:
an inner layer made of PTFE; and
an intermediate layer that is provided on an outer periphery of the inner layer and is made of PAE.

17. The catheter according to claim 1, wherein the hollow shaft comprises:
an inner layer made of PTFE; and
an intermediate layer that is provided on an outer periphery of the inner layer and is made of PAE.

18. The catheter according to claim 12, wherein the width of the linear member gradually decreases from the inner side of the linear member to the outer side of the linear member.
